(19) United States
(12) Patent Application Publication
(10) Pub. No.: US 2025/0127834 A1
(43) Pub. Date: Apr. 24, 2025

(54) PLANT-DERIVED COMPOSITIONS THAT MODULATE CELLULAR IMMUNITY

(71) Applicant: 4Life Patents, LLC, Sandy, UT (US)

(72) Inventor: David Vollmer, South Jordan, UT (US)

(21) Appl. No.: 18/922,318

(22) Filed: Oct. 21, 2024

Related U.S. Application Data
(60) Provisional application No. 63/544,930, filed on Oct. 19, 2023.

(51) Int. Cl. A61K 36/31; A61K 38/17

(52) U.S. Cl. CPC A61K 36/31; A61K 38/1729

(57) ABSTRACT

A composition that modulates cellular immunity in animals includes an ingredient derived from a plant. Such an ingredient may be referred to as “plant-derived” or “plant-based.” The plant-derived ingredient modulates cellular immunity in animals. Such a plant-derived ingredient may include an extract, fraction, or isolate from a seed of a plant, such as a seed of a plant from the family Brassicaceae. A method for modulating cellular immunity in an animal includes administering a plant-derived composition to the animal in amount that will cause the animal to elicit a cell-mediated immune response.

### CROSS-REFERENCE TO RELATED APPLICATION

A claim for priority is hereby made to the Oct. 19, 2023 filing date of U.S. Provisional Patent Application No. 63/544,930, titled PLANT-BASED COMPOSITIONS THAT MODULATE CELLULAR IMMUNITY, the entire disclosure of which is hereby incorporated herein.

### TECHNICAL FIELD

This disclosure relates generally to compositions that modulate cellular immunity in animals. More specifically, this disclosure relates to plant-derived, or plant-based, compositions that modulate cellular immunity in animals. This disclosure also relates to methods for modulating cellular immunity in animals, including, but not limited to, methods for modulating cell-mediated immunity, such as methods for eliciting cell-mediated immune responses.

### RELATED ART

The immune systems of vertebrates are equipped to recognize and defend the body from invading pathogenic organisms, such as viruses, bacteria, fungi, and parasites. Vertebrate immune systems typically include a cellular component and a noncellular component. The cellular component of an immune system includes the so-called “lymphocytes,” or white blood cells, of which there are several types. It is the cellular component of a mature immune system that typically mounts a primary, nonspecific response to invading pathogens, as well as being involved in a secondary, specific response to pathogens.

In the primary, or initial, response to an infection by a pathogen, some types of white blood cells locate and attack the invading pathogens. One type of such a white blood cell is the natural killer (NK) cell. Natural killer cells detect and destroy cells that have been infected with pathogens (e.g., viruses, etc.) and cancer cells. In addition, in response to detecting infected cells or cancer cells, natural killer cells produce and excrete chemicals to enlist other cells of the immune system in the attack of the infected or cancerous cells.

Only if an infection by invading pathogens continues to elude the primary immune response is a specific, secondary immune response to the pathogen needed. As this secondary immune response is typically delayed, it is also known as “delayed-type hypersensitivity.” A mammal, on its own, will typically not elicit a secondary immune response to a pathogen until about seven (7) to about fourteen (14) days after becoming infected with the pathogen. The secondary immune response is also referred to as an acquired immunity or as an adaptive immunity to specific pathogens. Pathogens have one or more characteristic proteins, which are referred to as “antigens.” In a secondary immune response, white blood cells known as B lymphocytes, or “B-cells,” and T lymphocytes, or “T-cells,” “learn” to recognize one or more of the antigens of a pathogen. The B-cells and T-cells work together to generate proteins called “antibodies,” which are specific for (e.g., configured to bind to or otherwise “recognize”) one or more certain antigens on a pathogen.

The T-cells are primarily responsible for the secondary, or delayed-type hypersensitivity, immune response to a pathogen or antigenic agent. There are three types of T-cells: T-helper cells, T-suppressor cells, and antigen-specific T-cells, which are also referred to as cytotoxic (meaning “cell-killing”) T-lymphocytes (CTLs) or as T-killer cells. The T-helper and T-suppressor cells, while not specific for certain antigens, perform conditioning functions (e.g., the inflammation that typically accompanies an infection) that assist in the removal of pathogens or antigenic agents from an infected host.

Antibodies, which make up a part of the noncellular component of an immune system, recognize specific antigens and, thus, are said to be “antigen-specific.” The generated antibodies then basically assist the white blood cells in locating and eliminating the pathogen from the body. Typically, once a white blood cell has generated an antibody against a pathogen, the white blood cell and all of its progenitors continue to produce the antibody. After an infection is eliminated, a small number of T-cells and B-cells that correspond to the recognized antigens are retained in a “resting” state. When the corresponding pathogenic or antigenic agents again infect the host, the “resting” T-cells and B-cells activate and, within about forty-eight (48) hours, induce a rapid immune response. By responding in this manner, the immune system mounts a secondary immune response to a pathogen, the immune system is said to have a “memory” for that pathogen.

Mammalian immune systems are also known to produce smaller proteins, known as “transfer factors,” as part of a secondary immune response to infecting pathogens. Transfer factors are another noncellular part of a mammalian immune system. Antigen-specific transfer factors are believed to be structurally analogous to antibodies, but on a much smaller molecular scale. Both antigen-specific transfer factors and antibodies include antigen-specific sites. In addition, both transfer factors and antibodies include highly conserved regions that interact with receptor sites on their respective effector cells. In transfer factor and antibody molecules, a third, “linker,” region connects the antigen-specific sites and the highly conserved regions.

Transfer factor is a low molecular weight isolate of lymphocytes. Antigen-specific transfer factors include antigen-specific regions that are believed to comprise about eight (8) to about twelve (12) amino acids. A second highly-conserved region of about ten (10) amino acids is thought to be a very high-affinity T-cell receptor binding region. The remaining amino acids may serve to link the two active regions or may have additional, as yet undiscovered properties. The antigen-specific region of a transfer factor molecule, which is analogous to the known antigen-specific structure of an antibody, but on a much smaller molecular weight scale, appears to be hyper-variable and is adapted to recognize a characteristic protein on one or more pathogens. The inducer and immune suppressor fractions are believed to impart transfer factor with its ability to condition the various cells of the immune system so that the cells are more fully responsive to the pathogenic stimuli in their environment.

Narrowly, transfer factors may have specificity for single antigens. U.S. Pat. Nos. 5,840,700 and 5,470,835, both of which issued to Kirkpatrick et al. (hereinafter collectively referred to as “the Kirkpatrick Patents”), disclose the isolation of transfer factors that are specific for certain antigens. More broadly, “specific” transfer factors have been generated from cell cultures of monoclonal lymphocytes. Even if these transfer factors are generated against a single pathogen, they have specificity for a variety of antigenic sites of that pathogen. Thus, these transfer factors are said to be “pathogen-specific” rather than antigen-specific. Similarly, transfer factors that are obtained from a host that has been infected with a certain pathogen are pathogen-specific.

Additionally, it is believed that pathogen-specific transfer factors may cause a host to elicit a delayed-type hypersensitivity immune response to pathogens for which such transfer factor molecules are not specific. Transfer factor “draws” at least the non-specific T-cells, the T-inducer and T-suppressor cells, to an infecting pathogen or antigenic agent to facilitate a secondary, or delayed-type hypersensitivity, immune response to the infecting pathogen or antigenic agent.

The immune system of a newborn has typically not developed, or “matured,” enough to effectively defend the newborn from invading pathogens. Moreover, prior to birth, many mammals are protected from a wide range of pathogens by their mothers. Thus, many newborn mammals cannot immediately elicit a secondary response to a variety of pathogens. Rather, newborn mammals are typically given secondary immunity to pathogens by their mothers. One way in which mothers are known to boost the immune systems of newborns is by providing the newborn with a set of transfer factors.

Transfer factor basically transfers the mother's acquired, specific (i.e., delayed-type hypersensitive) immunity to the newborn. This transferred immunity typically conditions the cells of the newborn's immune system to react against pathogens in an antigen-specific manner, as well as in an antigen- or pathogen-nonspecific fashion, until the newborn's immune system is able on its own to defend the newborn from pathogens. Thus, when transfer factor is present, the immune system of the newborn is conditioned to react to pathogens with a hypersensitive response, such as that which occurs with a typical delayed-type hypersensitivity response. Accordingly, transfer factor is said to “jump start” the responsiveness of immune systems to pathogens.

In mammals, transfer factor is provided by a mother to a newborn in colostrum, which is typically replaced by the mother's milk after a day or two. Conventionally, transfer factor has been obtained from the colostrum of milk cows, such as by the method described in U.S. Pat. No. 4,816,563 to Wilson et al. (hereinafter “Wilson”). In birds, transfer factor is provided by a mother to a developing chick in the egg, as described by U.S. Pat. No. 6,468,534 to Hennen et al. (hereinafter “Hennen”).

A variety of nutritional supplements based on colostrum and/or egg have been developed to provide the potential immune-modulating benefits of transfer factor to others. Despite the wide-spread use of colostrum-based and egg-based supplements and the significant beneficial effects of transfer factor, many people have been unable to benefit from transfer factor out of necessity (e.g., allergies, etc.) or a choice not to consume animal-based products.

### SUMMARY

This disclosure relates to plant-based compositions that may provide health benefits that are similar to or the same as the benefits that may be provided by transfer factor. For example, a plant-based composition of this disclosure may modulate cell-mediated in a subject (e.g., a mammal, such as a human, a domesticated pet, etc.). Thus, such a plant-based composition may be referred to as an “immune modulator.”

In one aspect, a composition for supporting immune health in a subject (e.g., a mammal, etc.) is disclosed. Such a composition includes a processed plant product with at least one protein that may modulate cell-mediated immunity in the subject. For example, the at least one protein may enable the subject's immune system to initiate a cell-mediated immune response more quickly, more efficiently, and/or more effectively than the cell-mediated response would be without the at least one protein. Stated another way, the at least one protein may elicit a cell-mediated immune response in the subject. More specifically, a dose of the composition may include the at least one protein in an amount that will elicit a desired response, such as the modulation of cell-mediated immunity or the elicitation of a cell-mediated immune response in the subject. In addition to the at least one protein, such a composition may include a plant-based capsule. In some embodiments, the composition may consist of plant-based elements or ingredients (e.g., the processed plant product, the capsule, etc.).

The processed plant product may comprise a processed portion of a plant that includes a sufficient amount of at least one protein that may modulate an immune response in a subject (e.g., a mammal, etc.). Without limitation, the processed plant product may comprise a processed plant seed product. In a specific embodiment, the process plant product may comprise a byproduct of vegetable oil (e.g., canola oil, or rapeseed oil, production, sunflower seed oil production, etc.). In further embodiments, the processed plant product may comprise an extract of the processed portion of the plant or even a fraction of the extract. As an example, the processed plant product may be fractionated to provide an upper molecular weight cutoff (UMWCO) (e.g., an UMWCO of about 30 kDa, an UMWCO of 30 kDa, an UMWCO of about 3 kDa, an UMWCO of 3 kDa, etc.). Such a processed plant product may be further fractionated to include a lower molecular weight cutoff (LMWCO) (e.g., a LMWCO of about 3 kDa, a LMWCO of 3 kDa, etc.).

The plant from which the processed plant product is obtained may comprise any plant that provides a suitable amount of at least one protein that may modulate an immune response in a subject (e.g., a mammal, etc.). For example, the processed plant product may be obtained from a plant in the family Brassicaceae. Examples of plants in the family Brassicacea include plants from the genus Raphanus (e.g., Raphanus sativus, etc.) and plants from the genus Brassica (e.g., Brassica juncea, Brassica napus, etc.), as well as plants from other families (e.g., Helianthus annuus, Cannabis sativa, etc.).

The at least one protein may comprise a so-called “storage protein” or “seed storage protein” (e.g., in embodiments where the plant product comprises a processed plant seed product). The at least one protein may comprise a plant defense-related protein, such as one or more defensins. Alternatively or in addition, the at least one protein may comprise one or more napins, cruciferins, and/or the like.

A plant-based composition of this disclosure may be a nutritional supplement that includes plant-based immune modulators, which may provide health benefits that are similar to or the same as the benefits that may be provided by transfer factor. In some embodiments, such a nutritional supplement may lack animal-based products (e.g., it may be acceptable for use by vegetarians, it may be acceptable for use by vegans, etc.). In other embodiments, such a nutritional supplement may include one or more plant-based immune modulators, as well as one or more animal-based immune modulators.

In addition to including a plant-based immune modulator and an optional animal-based immune modulator, a nutritional supplement according to this disclosure may include other ingredients, such as vitamins, minerals, proteins, polysaccharides, and/or botanicals (e.g., herbs, mushrooms, roots, etc.). Polysaccharides (e.g., beta glucans, mushroom extracts, etc.) are believed to provide further synergy in the effectiveness of a nutritional supplement of this disclosure.

In another aspect, a method for modulating cell-mediated immunity in a subject, such as a mammal (e.g. a human, a domesticated pet, etc.), is disclosed. The modulation of cell-mediated immunity may including eliciting a cell-mediated immune response in the subject, strengthening the subject's cell-mediated immunity, supporting the subject's cell-mediated immunity, or the like.

Such a method may include administering to the subject a composition that includes at least one protein that may modulate cell-mediated immunity in the subject. The composition that is administered to the subject may comprise a composition of this disclosure. The amount of the at least one protein in the composition (e.g., a dose of the composition, etc.) may be sufficient to modulate cell-mediated immunity in the subject (e.g., support cellular immunity, enhance cellular immunity, etc.). For example, the amount of the at least one protein in the composition (e.g., a dose of the composition, etc.) may be sufficient to elicit a cell-mediated immune response in a subject.

Other aspects of the disclosed subject matter, as well as features and advantages of various aspects of the disclosed subject matter, should be apparent to those of ordinary skill in the art through consideration of the ensuing description, the accompanying drawings, and the appended claims.

### BRIEF DESCRIPTION OF THE DRAWINGS

In the drawings:

FIG. 1 is a graph showing the results of a peripheral blood mononuclear cell (PBMC) cytotoxicity assay showing the effectiveness of aqueous extracts (AE) of two processed plant products (P00303 (processed seed from radishes (Raphanus sativus)) and P00397 (processed seed from mustard greens (Brassica juncea)) in inducing natural killer (NK) cells of the PBMCs to kill cancerous human lymphoblast cells (K562 cells) relative to the abilities of bovine colostrum (transfer factor (TF)) and interleukin-2 (IL-2) to induce NK cells to kill the K562 cells;

FIG. 2 is a graph showing the concentration of proteins in the AEs of the processed plant products (P00303 and P00397) and the concentration of proteins in bovine colostrum (TF), as determined with a Bradford assay;

FIG. 3 is an image of an SDS-PAGE (sodium dodecylsulfate-polyacrylamide gel electrophoresis) gel showing the protein content of various extracts of the processed plant products (P00303 and P00397);

FIG. 4 is a table showing the yields from C18 high-performance liquid chromatrography (HPLC) column fractionation of the processed plant products (P00303 and P00397) to provide five (5) extracts of each processed plant product (WA, WA-50ME, 50ME, 50-100ME, and 100ME);

FIG. 5 is a graph showing the results of a PBMC cytotoxicity assay showing the effectiveness of the extracts identified in the table of FIG. 4 in inducing NK cells to kill K562 cells relative to the abilities of TF and IL-2 to induce NK cells to kill 562 cells;

FIG. 6 is a table showing the yields from fractionation of the processed plant products (P00303 and P00397) with 30 kDa and 3 kDa filters;

FIG. 7 is an image of an SDS-PAGE gel showing the protein content of each fraction identified in the table of FIG. 6;

FIGS. 8A and 8B are graphs showing the results of a PBMC cytotoxicity assay showing the effectiveness of the fractions identified in the table of FIG. 6 in inducing NK cells to kill K562 cells relative to the abilities of TF and IL-2 to induce NK cells to kill K562 cells;

FIGS. 9A and 9B are dose curves showing the cytotoxicity of various concentrations of two of the processed plant products (P00303 and P00397), as normalized to a negative control (NC) and IL-2;

FIG. 10 is a graph showing the results of a PBMC cytotoxicity assay showing the effectiveness of two doses (0.78 μg/mL and 3.13 μg/mL) and a dose of a fraction (3.13 μg/mL of 3-30 kDa) of the processed plant products (P00303 and P00397) in inducing NK cells to kill K562 cells relative to the abilities of TF, a negative control (Lysis buffer), and IL-2 to induce NK cells to kill 562 cells;

FIGS. 11 and 12 are tables identifying the proteins in the 3 kDa to 30 kDa fractions of the aqueous extracts (AEs) of radish (Raphanus sativus) seeds (P00303) and mustard green (Brassica juncea) seeds (P00397), respectively.

### DETAILED DESCRIPTION

A composition of this disclosure may comprise a processed plant product from any of a variety of plants. Two specific plant products—seed from radishes (Raphanus sativus) and seed from mustard greens (Brassica juncea)—were processed, extracted, and evaluated to determine the potential efficacy of each of these processed plant products in supporting immune health in a subject (e.g., a mammal, etc.). More specifically, each of the processed plant products was evaluated to determine whether it includes at least one protein may elicit a cell-mediated immune response in the subject.

Each plant product was processed by grinding the plant product to a powder. An aqueous extract (AE) of each processed plant product was prepared by loading the powder (20 g) of each plant product into 100 ml stainless steel tubes. Each plant product was extracted twice with an organic solvent mixture of methylene chloride/methanol in a ratio of 1:1 using an ASE 300 automatic extractor at a temperature of 80° C. and pressure of 1,500 psi. Each extract solution was automatically filtered and collected. The powder of each plant product was then flushed with fresh solvent and purged with nitrogen gas to dry the powder. The powder of each plant product was then subjected to aqueous extraction at a temperature of 50° C. The aqueous solution was then filtered and freeze-dried to provide each aqueous extract (AE).

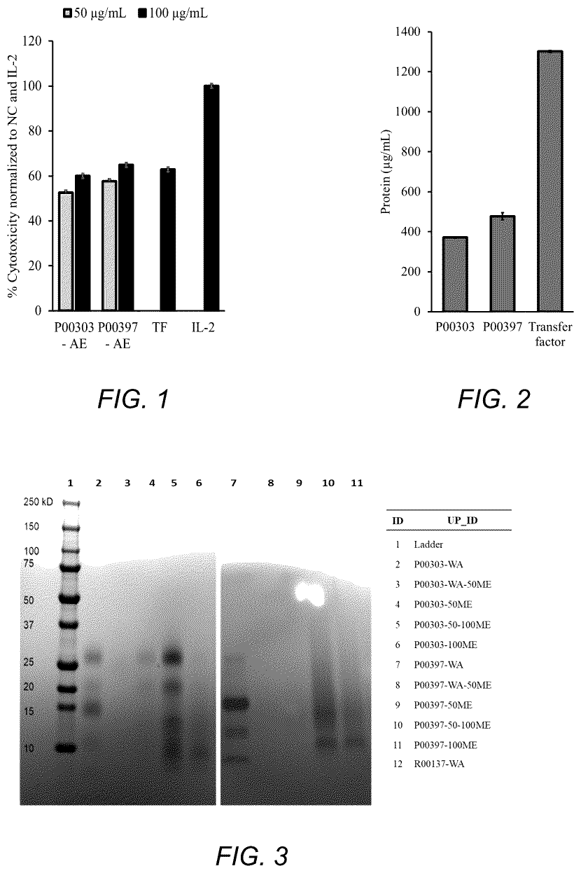

FIG. 1 is a graph showing the results of a peripheral blood mononuclear cell (PBMC) cytotoxicity assay showing the effectiveness of the AEs of the processed plant products of the radish seeds (P00303) and mustard green seeds (P00397) in inducing natural killer (NK) cells to kill cancerous human lymphoblast cells (K562 cells) relative to the abilities of bovine colostrum (TF) and interleukin-2 (IL-2) to induce NK cells to kill the K562 cells.

Specifically, a plate (e.g., a 96-well plate, etc.) was prepared with wells including about 2.5×10⁵ cells/well. The following samples were added to wells of the plate: (1) 50 μg/mL of the radish seed AE; (2) 100 μg/mL of the radish seed AE; (3) 50 μg/mL of the mustard green seed AE; (4) 100 μg/mL of the mustard green seed AE; (5) the IL-2 (20 μg/mL); (6) the TF (500 μg/mL); and (7) lysis buffer (i.e., no treatment). The PMBCs without treatment (sample 7) were used as a negative control. The IL-2 (sample 5) was used as a positive control. The TF (sample 6) was used as a reference.

The next day, 1×10⁶ K562 cells (human cancerous lymphoblasts) were suspended in 2.5 ml of cell culture medium and 2.5 ml of 10 μM Calcein-AM in a 15 ml conical tube, which was sealed and inverted to mix the K562 cells, the cell culture medium, and the Calcein-AM. The mixture was then incubated at 370 C for 30 minutes. The mixture was then centrifuged at 3,000 rpm for five minutes. The pellet was then resuspended in fresh cell culture medium. The process of centrifugation and resuspension occurred three times to wash out any unbound Calcein-AM.

The K562 cells were added to the wells of the plate to which samples had been added at a density of about 1×10⁴ cells/well (i.e., a 25:1 effector:target cell ratio, or PBMC:K562 cell ratio). The plate was centrifuged at 600 rpm for two minutes to gently settle the suspended cells on the bottoms of the wells. The plates were then scanned every hour for four hours. Scanning was conducted with an ImagExpress Pico by Molecular Devices to detect green fluorescence from the Calcein-AM. The parameters were adjusted to detect single, bright green cells. The number of live cells was calculated at each time point to provide a measure of cytotoxicity at each time point. The number of surviving K562 cells was normalized to the negative control and the IL-2 positive control (set to 100% cytotoxicity) to reduce the variation among individual experiments.

As shown in FIG. 1, the AEs of the radish seeds (P00303) and the mustard greens (P00397) were about as effective as colostrum (TF) in inducing natural killer (NK) cells to kill the cancerous human lymphoblast cells (K562 cells). More specifically, the colostrum (TF) (sample 6) exhibited a cytotoxicity of about 62%, while the 50 μg/mL AE of radish seeds (P00303) (sample 1) exhibited a cytotoxicity of about 52%, the 100 μg/mL AE of radish seeds (P00303-AE) (sample 2) exhibited a cytotoxicity of about 60%, the 50 μg/mL AE of mustard green seeds (P00397) (sample 3) exhibited a cytotoxicity of about 57%, and the 100 μg/mL AE of mustard green seeds (P00397) (sample 4) exhibited a cytotoxicity of about 66%.

Turning now to FIG. 2, Bradford assays were performed on the AEs of the plant products (i.e., the radish seeds (P00303) and the mustard green seeds (P00397)), as well as on the bovine colostrum (TF). In the Bradford assay, Bradford Reagent (Bio-Rad, 5000202) was added to wells of a plate at a volume of 250 μl/well. Five microliters (5 μl) of a sample (i.e., of the AE of one of the plant products, of the bovine colostrum (TF)) or of a reference standard for the Bradford assay was added to the 250 μl of Bradford Reagent in each well. The plate was then incubated at room temperature for five minutes before reading the absorbance at 595 nm of the wells of the plate.

From the absorbance measurements, the protein concentrations of the reference standards and the AEs of the three plant products were then calculated using a Bovine Serum Albumin (BSA) standard curve. As shown in FIG. 2, bovine colostrum (TF) had a protein concentration of about 1,300 μg/ml, while the AE of the radish seeds (P00303) had a protein concentration of about 380 μg/ml and the AE of the mustard green seeds (P00397) had a protein concentration of about 480 μg/ml.

FIG. 3 is an image of an SDS-PAGE (sodium dodecylsulfate-polyacrylamide gel electrophoresis) gel showing the protein content of the AEs of the radish seeds (P00303) and mustard green seeds (P00397) and the protein content of the bovine colostrum (TF). The samples were prepared for SDS-PAGE by placing each sample in SDS sample buffer (100 mM Tris, pH 6.8, 20% glycerol, 4% SDS 0.05% bromophenol blue, and 10% β-mercaptoethanol) and boiling the SDS sample buffer and the sample for 5 minutes.

A 5-15% Tris-Tricine gradient gel was prepared. One lane of the gel was loaded with molecular weight (MW) markers. Other lanes of the gel were loaded with samples. Electrophoresis was then conducted. The gel was stained with Coomassie (45% methanol, 10% glacial acetic acid, 0.25% Coomassie G-250) nutating for one-hour at room temperature, and then destained three times for 30 minutes each with 45% methanol, 10% glacial acetic acid. The gel was then visualized on a ThermoFisher iBright gel and Western blot documentation station. FIG. 3 is an image of the gel, showing the MW markers (to the left of lane 1) and the proteins that were present in the AE of the radish seeds (P00303) (lane 1), the AE of the mustard green seeds (P00397) (lane 2), and the bovine colostrum (TF) (lane 12).

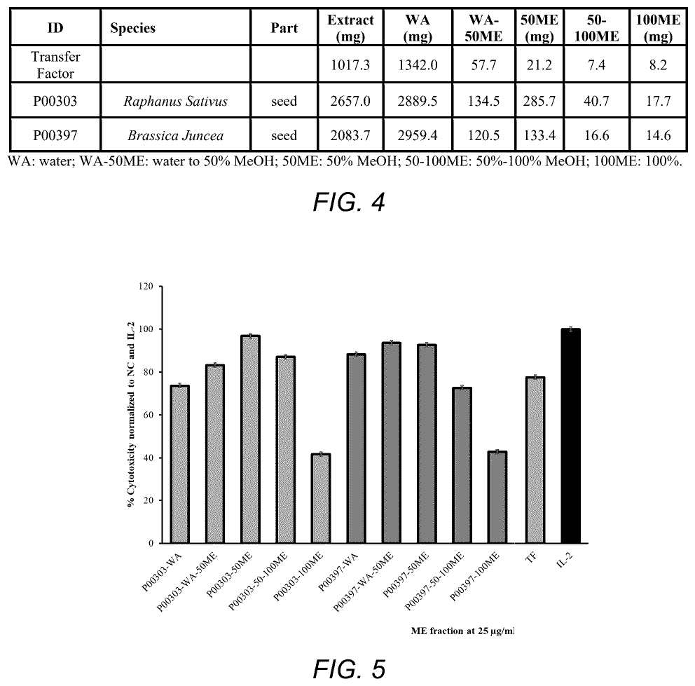

FIG. 4 is a table showing the yields from C18 high-performance liquid chromatrography (HPLC) column fractionation of the AE of the radish seeds (P00303), the AE of the mustard green seeds (P00397), and the bovine colostrum (Transfer Factor).

The bovine colostrum and each AE was dissolved in 13 mL DI water and 2 mL DMSO to improve solubility. Each solution was then loaded onto a pre-packed Biotage® Sfar C18 column (Duo 100 A 30 μm, 60 g) and pushed into the column bed. Liquid dripping from the column was collected into a waste beaker until the solution just reached the frit of the waste beaker. The column then was eluted with a gradient with methanol in water as follows: 2 column volumes (CV) of 100% distilled (DI) water (WA); 1.4 CV of 100% DI water to 50% methanol (WA-50ME); 1.3 CV of 50% methanol (50ME); 1.6 CV of 50% methanol to 100% methanol (50-100ME); 2 CV of and 100% methanol (100ME). The elution was collected, with each of the five column fractions WA, WA-50ME, 50ME, 50-100ME, and 100 ME) being separate from the other column fractions. Each of the five column fractions was then dried (e.g., by evaporating solvents (e.g., with a rotary evaporator, etc.), etc.). Each column fraction was then weighed, providing the results set forth in the table of FIG. 4.

The five column fractions for the AE of the radish seeds (P00303) and the AE of the mustard green seeds (P00397) were then tested for PBMC cytotoxicity. Specifically, a plate (e.g., a 96-well plate, etc.) was prepared with wells including about 2.5×10⁵ PBMC cells/well, as described in reference to FIG. 1. The following samples were added to cells of the plate: (1) 25 μg/mL of the WA column fraction of the AE of the radish seeds (P00303); (2) 25 μg/mL of the WA-50ME column fraction of the AE of the radish seeds (P00303); (3) 25 μg/mL of the 50 ME column fraction of the AE of the radish seeds (P00303); (4) 25 g/mL of the 50-100ME column fraction of the AE of the radish seeds (P00303); (5) 25 g/mL of 100 ME column fraction of the AE of the radish seeds (P00303); (6) 25 μg/mL of the WA column fraction of the AE of the mustard green seeds (P00397); (7) 25 μg/mL of the WA-50ME column fraction of the AE of the mustard green seeds (P00397); (8) 25 μg/mL of the 50 ME column fraction of the AE of the mustard green seeds (P00397); (9) 25 μg/mL of the 50-100ME column fraction of the AE of the mustard green seeds (P00397); (10) 25 μg/mL of 100 ME column fraction of the AE of the mustard green seeds (P00397); (11) 500 μg/mL of the bovine colostrum (TF); (12) 20 μg/mL of the IL-2; and (13) lysis buffer (i.e., no treatment). The PMBCs without treatment (sample 13) were used as a negative control. The TF (sample 11) was used as a reference. The IL-2 (sample 12) was used as a positive control.

K562 cells were prepared, added to the wells of the plate to which samples had been added, incubated, and processed in the manner described in reference to FIG. 1. The number of surviving K562 cells in each well was normalized to the negative control and the IL-2 positive control (set to 100% cytotoxicity) to reduce the variation among individual experiments. The data obtained from this PBMC cytotoxicity assay are illustrated by FIG. 5.

As FIG. 5 shows, measurable activity was observed in each of the wells and, thus, for each of the column fractions. For example, it was found that, the 50ME column fraction of the AE of the radish seeds (P00303) had the highest activity of all of the radish seed (P00303) column fractions, while the 100ME column fraction of the AE of the radish seeds (P00303) showed the least activity of all of the radish seed (P00303) column fractions. In the case of the AE of the mustard green seeds (P00397), the highest activity was found in the WA-50ME and 50ME column fractions, while the least activity was observed in the 100ME column fraction.

Further analysis was conducted by subjecting each of the column fractions identified in the table of FIG. 4 and the graph of FIG. 5 to ultrafiltration. Specifically, a 250 mg sample from each column fraction and a 250 mg sample of bovine colostrum (TF) were each dissolved in 150 ml of DI water. For each of these reconstituted column fractions and reconstituted bovine colostrum, each filter (i.e., a 30 kDa membrane disc and a 3 kDa membrane disc) used in the ultrafiltration process was rinsed by floating its skin (glossy) side down in a beaker filled with DI water and sonicating the beaker. During this process, the water was changed three times (e.g., about every 15 minutes).

During a first filtration, the 30 kDa membrane disc was placed in an ultrafiltration device with the skin (glossy) side up. The reconstituted column fraction/reconstituted bovine colostrum was transferred into the ultrafiltration device and nitrogen gas was applied. The <30 kDa fraction of the reconstituted column fraction passed through the 30 kDa membrane disc.

Once the first filtration of the reconstituted column fraction/reconstituted bovine colostrum was complete, the membrane was removed and placed in a beaker with about 250 ml of DI water. The 30 kDa membrane disc was sonicated for about 1 hour and the fraction was kept as >30 kDa fraction of the column fraction.

During a second filtration, the 3 kDa membrane disc was placed in the ultrafiltration device with the skin (glossy) side up. The <30 kDa fraction was transferred into the ultrafiltration device and nitrogen gas was applied. The <3 kDa fraction of the <30 kDa fraction passed through the 3 kDa membrane disc.

Once the second filtration (i.e., ultrafiltration of the <30 kDa fraction) was complete, the 3 kDa membrane disc was removed and placed in a beaker with about 250 mL of DI water. The 3 kDa membrane disk was sonicated for about 1 hour and the fraction was kept as 3-30 kDa fraction. The filtrate solution that passed through the 3 kDa membrane was labeled as <3 kDa fraction. All the ultrafiltration fractions were freeze-dried to remove water, resulting in a powdered molecular weight fraction.

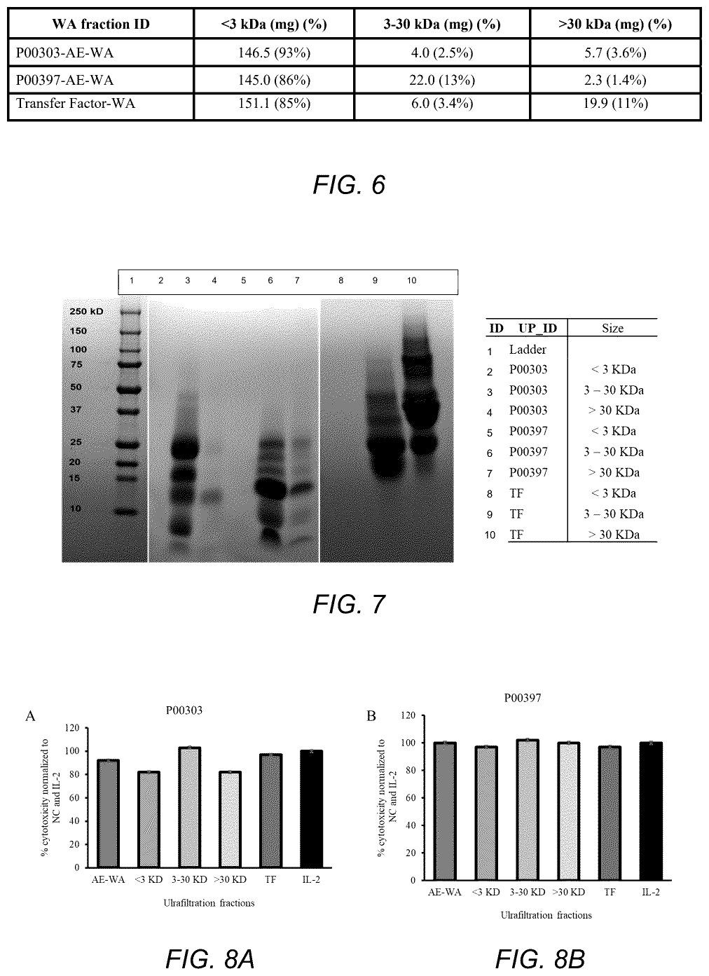

The table of FIG. 6 shows the <3 kDa, 3 kDa to 30 kDa, and >30 kDa yields from filtration of each of the AE of the radish seeds (P00303), the AE of the mustard green seeds (P00397), and the bovine colostrum (TF).

The filtration fractions were subjected to SDS-PAGE, as described in reference to FIG. 3. FIG. 7 is an image of an SDS-PAGE gel showing the protein content of each of the filtration fractions of the AE of the radish seeds (P00303), the filtration fractions of the AE of the mustard green seeds (P00397), and the filtration fractions of the colostrum (TF).

PBMC cytotoxicity assays, as described in reference to FIG. 1, were then conducted for each filtration fraction (i.e., <3 kDa, 3 kDa-30 kDa, >30 kDa) of the AE of the radish seeds (P00303) and the AE of the mustard green seeds (P00397), with bovine colostrum (TF) also being tested as a reference and IL-2 being tested as a positive control. Specifically, a plate (e.g., a 96-well plate, etc.) was prepared with wells including about 2.5×10⁵ PBMC cells/well, as described in reference to FIG. 1. The following samples were added to cells of the plate: (1) 12.5 μg/mL of the WA column fraction of the AE of the radish seeds (P00303); (2) 12.5 μg/mL of the <3 kDa filtration fraction of the AE of the radish seeds (P00303); (3) 12.5 μg/mL of the 3 kDa to 30 kDa filtration fraction of the AE of the radish seeds (P00303); (4) 12.5 μg/mL of the >30 kDa filtration fraction of the AE of the radish seeds (P00303); (5) 12.5 μg/mL of WA column fraction of the AE of the mustard green seeds (P00397); (6) 12.5 μg/mL of the <3 kDa filtration fraction of the AE of the mustard green seeds (P00397); (7) 12.5 μg/mL of the 3 kDa to 30 kDa filtration fraction of the AE of the mustard green seeds (P00397); (8) 12.5 μg/mL of the >30 kDa filtration fraction of the AE of the mustard green seeds (P00397); (9) 500 μg/mL of the bovine colostrum (TF); (10) 20 μg/mL of the IL-2; and (11) lysis buffer (i.e., no treatment). The 12.5 μg/mL concentration was chosen to discriminate between the filtration fractions, as it was determined that activity at higher concentrations could saturate the PBMC assay. The bovine colostrum (TF) (sample 9) was used as a reference control. The IL-2 sample (sample 10) was used as a positive control. The PMBCs without treatment (sample 11) were used as a negative control.

K562 cells were prepared, added to the wells of the plate to which samples had been added, incubated, and processed in the manner described in reference to FIG. 1. The number of surviving K562 cells in each well was normalized to the negative control and the IL-2 positive control (set to 100% cytotoxicity) to reduce the variation among individual experiments. FIG. 8 illustrates the data obtained from this PBMC cytotoxicity assay.

As shown by FIGS. 8A and 8B, for the AE of the radish seeds (P00303), the 3 kDa to 30 kDa filtration fraction had the highest activity in the PBMC cytotoxicity assay. The activity of that filtration fraction was better than the original AE of the radish seeds at the same concentration and was comparable to activities of the TF and the IL-2. All three filtration fractions of the AE of the mustard green seeds (P00397) showed comparable activity compared to the original AE of the mustard green seeds at the same concentration and comparable activity to the TF and the IL-2. These results indicate that the AEs of the radish seeds and the mustard green seeds and various filtration fractions (e.g., molecular weight fractions, etc.) of the AEs of the radish seeds (P00303) and mustard green seeds (P00397) are about as effective as or even more effective than bovine colostrum (e.g., transfer factor, etc.), and IL-2 in eliciting cell-mediated immunity, or a secondary immune response (e.g., by NK cells in the PBMC assay, etc.).

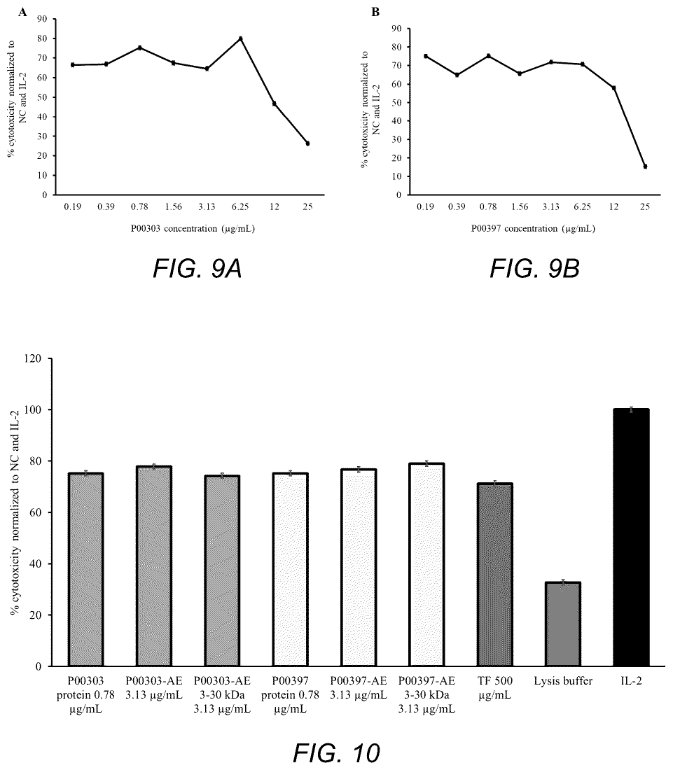

To confirm the activity of the AE of the radish seeds (P00303) and the AE of the mustard green seeds (P00397), proteins from raw seed materials were extracted and tested in PBMC cytotoxicity assays at eight concentrations (0.19 μg/mL, 0.39 μg/mL, 0.78 μg/mL, 1.56 μg/mL, 3.13 μg/mL, 6.25 μg/mL, 12 μg/mL, and 25 μg/mL), with lysis buffer as a negative control (NC) and IL-2 as a positive control. The results of the PBMC cytotoxicity assays conducted on the various concentrations of the AE of radish seeds (P00303) are shown in FIG. 9A and the results of the PBMC cytotoxicity assays conducted on the various concentrations of the AE of mustard green seeds (P00397) are shown in FIG. 9B. The higher concentrations are believed to interfered with the lysis buffer used in the PBMC cytotoxicity assays, resulting in less activity than the lower concentrations.

As FIGS. 9A and 9B show, the 0.78 μg/mL concentration of both the AE of the radish seeds (P00303) and the AE of the mustard green seeds (P00397) showed strong activity in inducing PBMCs to elicit an immune response against K592 cells. This concentration of each AE was chosen for comparison with the original AEs of the radish seeds (P00303) and the mustard green seeds (P00397) at concentrations of 3.13 μg/mL, 3.13 μg/mL concentrations of the 3 kDa to 30 kDa filtration fractions of the AEs of the radish seeds (P003103) and the mustard green seeds (P00397), colostrum (TF) at a concentration of 500 μg/mL, IL-2 at a concentration of 20 μg/mL, and lysis buffer.

As shown by the graph of FIG. 10, all of the test results were comparable to each other. More specifically, the effectiveness of the AEs of the radish seed (P00303) at inducing NK cells to kill K562 cells, or in eliciting a cell-mediated immune response, ranged from 74.2% to 77.8% of the effectiveness of IL-2, while the effectiveness of the AEs of the mustard green seed (P00397) at inducing NK cells to kill K562 cells, or at eliciting a cell-mediated immune response, ranged from 75.2% to 79.0% of the effectiveness of IL-2 at inducing NK cells to kill K562 cells, or at eliciting a cell-mediated immune response. Moreover, the AEs of the radish seeds (P00303) and the mustard green seeds (P00397) were more effective than colostrum (TF) at inducing NK cells to kill K562 cells, or at eliciting a cell-mediated immune response.

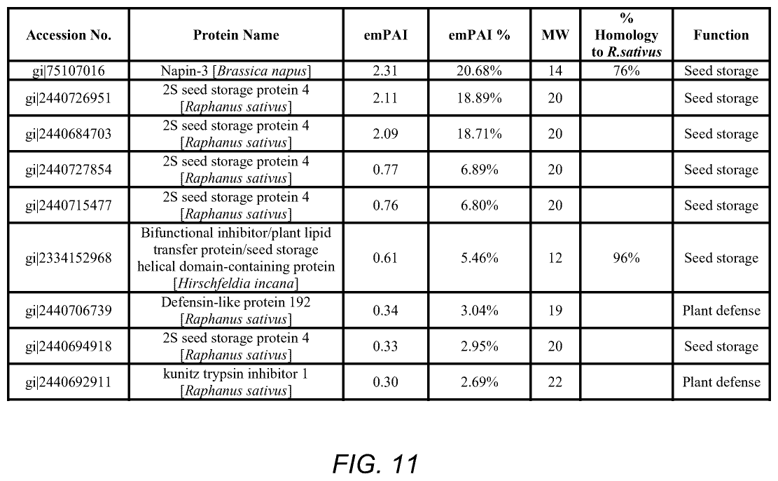

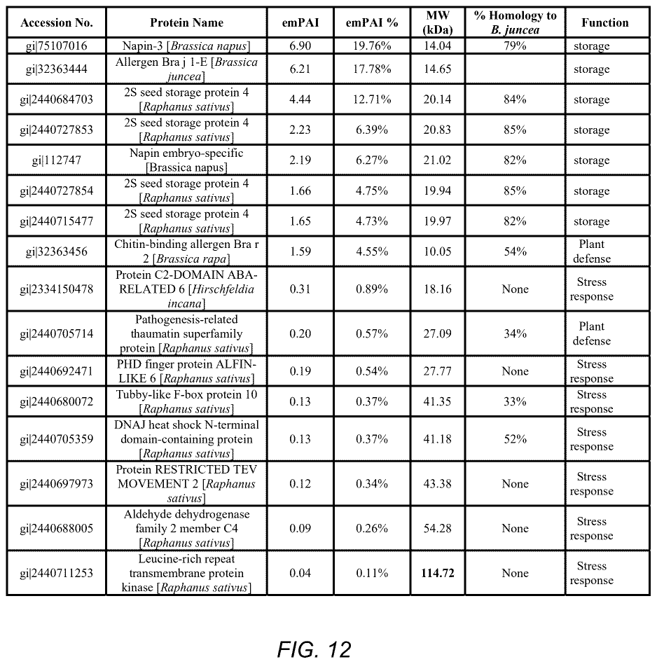

Liquid chromatography-tandem mass spectrometry (LC-MS/MS) and protein sequencing were carried out for the 3 kDa and 30 kDa filtration fraction of the AE of the radish (Raphanus sativus) seeds (P00303) to sequence proteins present in the 3 kDa to 30 kDa filtration fraction. The Brassicaceae taxonomy database from the National Center for Biotechnology Information at the National Institutes of Health was searched for homologous protein sequences. The proteins present in the 3 kDa to 30 kDa filtration fraction of the AE of the radish seeds (P00303) are identified in the table of FIG. 11. The exponentially modified protein abundance index (emPAI) and emPAI % were calculated for each of the proteins. Proteins that are not native to R. sativus were assessed for homology to similar proteins from R. sativus.

The 3 kDa to 30 kDa fraction of the AE of the radish (R. sativus) seeds (P00303) includes 17 proteins. As the table of FIG. 11 indicates, seven of the proteins are known seed storage proteins, with five of those identified from R. sativus sequences and two from related species of same genus plant with high homology to R. sativus. There is also one R. sativus Defensin protein that accounted for 3% of the total protein in the sample and a kunitz trypsin inhibitor, which is a protease that protects the seed proteins from being degraded by their environment. The remaining proteins, which are not listed in the table of FIG. 11, are low abundance; they include a cell wall adaptation protein, a cell signaling protein, and other proteins.

With returned reference to the image of the SDS-PAGE gel of FIG. 7, which shows proteins of the 3 kDa to 30 kDa filtration fraction of the AE of the radish seeds (R. sativus), seed storage proteins accounted for the bands at 12 kDa, 14 kDa, and 19-20 kDa, while kunitz trypsin inhibitor 1 was present in the band at 22-24 kDa.

LC-MS/MS and protein sequencing were also carried out for the 3 kDa and 30 kDa filtration fraction of the AE of the mustard green (Brassica juncea) seeds (P00397) to sequence proteins present in the 3 kDa to 30 kDa filtration fraction. The Brassicaceae taxonomy database was searched for homologous protein sequences. The proteins present in the 3 kDa to 30 kDa filtration fraction of the AE of the mustard green seeds (P00397) are identified in the table of FIG. 12. The emPAI and emPAI % were calculated for each of the proteins. Proteins that are not native to B. juncea were assessed for homology to similar proteins from B. juncea.

The 3 kDa to 30 kDa fraction of the AE of the mustard green seeds (B. juncea) seeds (P00397) includes 61 proteins. As the table of FIG. 12 indicates, the seven most abundant proteins are known seed storage proteins, Napin, and 2S seed storage proteins. The only protein found that is native to B. juncea is also a 2S seed storage protein. A number of plant defense and stress response genes are also present, including Chitin-binding allergen Bra r 2, which is involved in PAMP-triggered immunity (PTI) in plants. The others proteins are involved in defense against microorganisms or environmental stress.

Extracts, fractions (e.g., column fractions, filtration fractions, dialysates, etc.), and/or isolates from a variety of other sources are believed to provide similar results. For example, protein isolates from sunflower seeds (Helianthus annuus), hemp seeds (Cannabis sativa), and canola (i.e., rapeseed) seeds (Brassica napus) perform as well as or better than bovine colostrum in PBMC cytotoxicity assays.

Since the PMBCs used in the PBMC cytotoxicity assays are derived from healthy humans, it is reasonable for those of ordinary skill in the art to assume that plant-based immune modulators and compositions including plant-based immune modulators will have the same effects in vivo in humans and other animals.

A composition of this disclosure may include an extract (e.g., an AE, etc.), a fraction (e.g., a column fraction, a filtration fraction (e.g., a 3 kDa to 30 kDa filtration fraction, etc.), a dialysate, etc.), and/or an isolate of a part of a plant, or a plant product. The plant product may be a seed from the plant. In a specific embodiment, the composition may include an extract, fraction, and/or isolate of a seed from a plant of the family Brassicaceae (e.g., R. sativus, B. juncea, B. napus, etc.). The composition may include a combination of plant products. The composition may consist of plant-based ingredients or it may include plant-based ingredients.

The composition may include a dosage of the extract, fraction, or isolate of the plant product(s) that supports elicits cell-mediate immunity in a subject to which the composition is to be administered (e.g., a human, an animal, etc.).

Optionally, a composition of this disclosure may include one or more additives, which may be selected from vitamins, minerals, proteins, polysaccharides, botanicals, and the like.

A method for modulating cellular immunity in an animal includes administering a plant-derived composition to the animal in amount, or a dosage, that will support a cell-mediated immune response in an animal or cause the animal to elicit a cell-mediated immune response. Administration of the plant-derived composition may comprise enteral (i.e., oral) administration of the plant-derived composition. The plant-derived composition may be administered at regular intervals; for example, daily, twice daily, three times daily, four times daily, etc.

Although this disclosure provides many specifics, these should not be construed as limiting the scope of any of the claims that follow, but merely as providing illustrations of some embodiments of elements and features of the disclosed subject matter. Other embodiments of the disclosed subject matter, and of their elements and features, may be devised which do not depart from the spirit or scope of any of the claims. Features from different embodiments may be employed in combination. Accordingly, the scope of each claim is limited only by its plain language and the legal equivalents thereto.

What is claimed:

1. A composition for supporting immune health in a mammal, comprising:
a processed plant product including a protein that modulates cell-mediated immunity in a mammal.

2. The composition of claim 1, wherein the plant product includes a plurality of proteins that modulate cell-mediated immunity in mammals.

3. The composition of claim 1, wherein in the protein comprises a storage protein.

4. The composition of claim 1, wherein the protein comprises a defensin.

5. The composition of claim 1, wherein the processed plant product comprises a processed plant seed product.

6. The composition of claim 5, wherein the processed plant product comprises a processed product of a seed from family Brassicaceae.

7. The composition of claim 6, wherein the processed product comprises a processed product of a seed of a Brassica species of plant.

8. The composition of claim 7, wherein the processed product comprises a processed product of a seed from Brassica juncea or Raphanus sativus.

9. The composition of claim 6, wherein the processed product comprises a byproduct of vegetable oil production.

10. The composition of claim 1, wherein the processed plant product comprises an extract of a processed plant product.

11. The composition of claim 10, wherein the processed plant product comprises a fraction of the extract.

12. The composition of claim 10, wherein the processed plant product comprises a fraction with an upper molecular weight cutoff of about 30 kDa.

13. The composition of claim 12, wherein the processed plant product comprises a fraction with a lower molecular weight cutoff of about 3 kDa.

14. The composition of claim 1, wherein a dosage of the protein elicits cell-mediated immunity in the mammal.

15. The composition of claim 1, further comprising:
a plant-based capsule containing the processed plant product.

16. A method for eliciting cell-mediated immunity in a mammal, comprising:
administering a processed plant product including an extract, a fraction, or an isolate of a plant product that includes protein that modulates cell-mediated immunity in a mammal.

17. The method of claim 16, wherein the processed plant product comprises a processed plant seed.

18. The method of claim 17, wherein the processed plant seed comprises a processed seed of Raphanus sativus or Brassica juncea.

19. The method of claim 16, wherein administering comprises administering a dosage of the composition tailored to support cellular immunity by the mammal.

20. The method of claim 16, wherein administering comprises administering a dosage of the composition tailored to elicit cell-mediated immunity in the mammal.
